SEQUENCE LISTING (3 sequences)

SEQ ID NO: 1 - protein, 620 aa, Myceliophthora thermophila
MRSFISAATLLVGILTPSVAAAPPSTPEQRDLLVPITEREEAAVKARQQSCNTPSNRACWTDGYDINTDYEVDSPDTGVVRPYTLTLTEVDNWTGPDGVVKEKVMLVNNSIIGPTIFADWGDTIQVTVINNLETNGTSIHWHGLHQKGTNLHDGANGITECPIPPKGGRKVYRFKAQQYGTSWYHSHFSAQYGNGVVGAIQINGPASLPYDTDLGVFPISDYYYSSADELVELTKNSGAPFSDNVLFNGTAKHPETGEGEYANVTLTPGRRHRLRLINTSVENHFQVSLVNHTMTIIAADMVPVNAMTVDSLFLGVGQRYDVVIEASRTPGNYWFNVTFGGGLLCGGSRNPYPAAIFHYAGAPGGPPTDEGKAPVDHNCLDLPNLKPVVARDVPLSGFAKRPDNTLDVTLDTTGTPLFVWKVNGSAINIDWGRPVVDYVLTQNTSFPPGYNIVEVNGADQWSYWLIENDPGAPFTLPHPMHLHGHDFYVLGRSPDESPASNERHVFDPARDAGLLSGANPVRRDVTMLPAFGWVVLAFRADNPGAWLFHCHIAWHLEAGLGVVYLERADDLRGAVSDADADDLDRLCADWRRYWPTNPYPKSDSGLKHRWVEEGEWLVKA

SEQ ID NO: 2 - protein, 363 aa, Coprinopsis cinerea
MKLSLLSTFAAVIIGALALPQGPGGGGSVTCPGGQSTSNSQCCVWFDVLDDLQTNFYQGSKCESPVRKILRIVFHDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYRAAMAKMSVLGFDRNALTDCSDVIPSAVSNNAAPVIPGGLTVDDIEVSCPSEPFPEIATASGPLPSLAPAP

SEQ ID NO: 3 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

(19) United States
(12) Patent Application Publication
(10) Pub. No.: US 2018/0327794 A1
(43) Pub. Date: Nov. 15, 2018

(54) Enzymatic Epoxydation Of Natural Rubber

(71) Applicant: Novozymes A/S, Bagsvaerd (DK)

(72) Inventor: Suchitra Tripathy, Bangalore (IN)

(73) Assignee: Novozymes A/S, Bagsvaerd (DK)

(21) Appl. No.: 15/775,139

(22) PCT Filed: Nov. 21, 2016
(86) PCT No.: PCT/EP2016/078328

§ 371 Date: May 10, 2018

(30) Foreign Application Priority Data
Nov. 23, 2015 (IN) 6286/CHE/2015

(51) Int. Cl. C12P 17/02; C08C 19/06

(52) U.S. Cl. CPC C12P 17/02; C08C 19/06

(57) ABSTRACT

Disclosed is a method for epoxidating natural rubber using enzymes that generate reactive oxygen species.

### REFERENCE TO SEQUENCE LISTING

This application contains a Sequence Listing in computer readable form. The computer readable form is incorporated herein by reference.

### FIELD OF THE INVENTION

The present invention relates to a method for producing an epoxidized natural rubber, more specifically it relates to an enzymatic method for expoxidization of natural rubber.

### BACKGROUND OF THE INVENTION

Natural rubber, also called India rubber or caoutchouc, as initially produced, consists of polymers of the organic compound isoprene, with minor impurities of other organic compounds plus water. Malaysia is a leading producer of rubber. Forms of poly-isoprene that are used as natural rubbers are classified as elastomers. Natural rubber is used by many manufacturing companies for the production of rubber products. Currently, rubber is harvested mainly in the form of the latex from certain trees. The latex is a sticky, milky colloid drawn off by making incisions into the bark and collecting the fluid in vessels in a process called “tapping”. The latex then is refined into rubber ready for commercial processing. Natural rubber is used extensively in many applications and products, either alone or in combination with other materials. In most of its useful forms, it has a large stretch ratio and high resilience, and is extremely waterproof.

Conventional rubber products such as tyres contain epoxidized natural rubber. Typical epoxidization of natural rubber occurs through a series of steps of: collecting latex from rubber trees (for example, Hevea brasiliensis), concentrating the collected latex centrifugation; adding a surfactant to the resulting concentrated latex and subsequently adding formic acid while stirring; slowly introducing hydrogen peroxide over several hours and then allowing the epoxidation reaction to proceed for about one day; coagulating the resulting epoxidized natural rubber in latex form; and optionally neutralizing, water washing, and drying the coagulated natural rubber latex.

The production of epoxidized latex this way has the advantage that it allows uniform epoxidation of rubber because the rubber is epoxidized while having the same particle size (0.1 to several microns) as when it is present in the latex. However, the production cost of epoxidized natural rubber is very high due to the long reaction time, the use of expensive chemical agents and the large number of steps. Moreover, the rubber is destabilized such that it can easily coagulate; therefore, a surfactant needs to be added, which increases the cost and also creates other problems: for example, a reduction in the rubber physical properties of a final rubber product due to the absorption of moisture caused by the surfactant remaining in the product; and difficulties in controlling the temperature during the epoxidation reaction, which require the operator to continuously monitor the reaction. In addition, the use of surfactants, stabilizers and other harsh chemicals like formic acid is also not very sustainable for the environment.

### SUMMARY OF THE INVENTION

The invention provides a method for producing a partially or completely epoxidized rubber from natural rubber comprising:
a) contacting a natural rubber substrate with an enzyme composition that generates reactive oxygen species
b) recovering the epoxidised rubber.

The invention further discloses epoxidized rubber prepared according to the method of the invention.

The epoxidized rubber of the invention may be used for same application as epoxidized rubber prepared according to the conventional method, but represent an environment friendly alternative to the conventional product since the method can be performed faster and with use of less harsh chemicals compared with the conventional process.

### DETAILED DESCRIPTION OF THE INVENTION

The invention provides a method for producing completely or partially epoxidized rubber from natural rubber comprising:
(a) Contacting a natural rubber substrate with one or more enzyme(s) that generates reactive oxygen species in a time period until the desired degree of epoxidation is achieved;
(b) recovering the epoxidised rubber.

In comparison with the traditional chemical epoxidation of rubber the invention provides several benefits:
use of harsh chemicals like acetic acid or formic acid can be partially or completely eliminated;
the epoxidation of natural rubber can be carried out in the presence or absence of Hydrogen peroxide;
epoxidation can be carried out at natural pH of the rubber latex;
the reaction time of epoxidation process can be reduced; and
stabilizer used for maintaining colloidal stability of rubber latex during epoxidation process can be reduced or eliminated.

This is a greener technology compared to the conventional epoxidation process.

The natural rubber substrate may according to the invention be any such natural rubber substrate as known in are as long as it contains double bonds that can be epoxidize. Latex is a preferred natural rubber according to the invention.

The natural rubber substrate may be in any form wherein it is provided, such as solid or liquid form, where the liquid form is preferred. If the rubber substrate is in solid form the rubber substrate should preferably be in small particulate form to provide a large surface which will reduce the necessary reaction time to achieve a desired degree of epoxidation compare with the same substrate in a form with larger particles. This is all well known to the skilled person that a large surface provides for a faster reaction rate in such a non-homogeneous system.

The method of the invention takes place in an aqueous reaction mixture wherein the natural rubber is dispersed.

The one or more enzyme(s) that generate reactive oxygen species may according to the invention be selected among all enzymes known to provide such species. Preferably, the one or more enzyme(s) that generate reactive oxygen species is/are selected among laccases, peroxidases and carbohydrate oxidases, such as glucose oxidase.

In one embodiment the one or more enzyme(s) that generate reactive oxygen species is a laccase selected among laccases having an amino acid sequence identity of at least 80% identity, preferably at least 85% identity, preferably at least 90% identity, preferably at least 95% identity, preferably at least 96% identity, preferably at least 97% identity, preferably at least 98% identity, preferably at least 99% identity or 100% identity to the mature peptide of SEQ ID NO: 1.

In another embodiment the one or more enzyme(s) that generate reactive oxygen species is a peroxidase selected among peroxidases having an amino acid sequence identity of at least 80% identity, preferably at least 85% identity, preferably at least 90% identity, preferably at least 95% identity, preferably at least 96% identity, preferably at least 97% identity, preferably at least 98% identity, preferably at least 99% identity or 100% identity to the mature peptide of SEQ ID NO: 2.

In another embodiment the one or more enzyme(s) that generate reactive oxygen species is a glucose oxidases selected among glucose oxidases having an amino acid sequence identity of at least 80% identity, preferably at least 85% identity, preferably at least 90% identity, preferably at least 95% identity, preferably at least 96% identity, preferably at least 97% identity, preferably at least 98% identity, preferably at least 99% identity or 100% identity to the mature peptide of SEQ ID NO: 3.

The one or more enzyme(s) that generate reactive oxygen species may be added as pure enzyme, as an aqueous solution thereof or as an enzyme composition that comprises the one or more enzyme(s) that generate reactive oxygen species, where it is preferred to use enzyme compositions.

An enzyme composition is the typical product wherein commercial enzymes are supplied and may in addition to the active enzyme comprise further enzymes, solvents, diluents, stabilizers, fillers coloring agents etc.

Preferred enzyme compositions for use according to the invention include enzyme compositions such as Denilite IIS and Denilite COLD (Novozymes A/S, Bagsvaerd, Denmark) and Baysolex VPSP 20019 (Bayer AG, Leverkusen, Germany).

In some embodiments a mediator may be added to the reaction medium in order to facilitate the reactions. Mediators having the ability to accelerate an enzymatic oxidation reaction are known in the art and such mediators may also be used in the methods according to the present invention.

Enzymes that generate reactive oxygen species in general require oxygen to perform their intended reactions. The oxygen dissolved in the reaction mixture and that dissolves in the mixture during the incubation with the enzyme may be sufficient for achieving the intended degree of epoxidation, however, in some embodiments it may be beneficial to supply additional oxygen into the system to provide for a satisfactory reaction rate and a satisfactory degree of epoxidation. Oxygen may be supplied to the system using techniques known in the art, such as air sparging through the mixture, stirring, air bubbling through the mixture, addition of H₂O₂ optionally together with a catalase etc. A preferred way of supplying oxygen is addition of H₂O₂ optionally together with a catalase.

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (EC 1.11.1.10) catalyze formation of hypochlorite from chloride ions.

One preferred peroxidase for use according to the invention is the peroxidase having the sequence of the mature protein of SEQ ID NO: 2. In one embodiment the mature protein of SEQ ID NO: 2 corresponds the amino acids 19 to 363 of SEQ ID NO: 2.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

One preferred laccase for use according to the invention is the laccase having the sequence of the mature protein of SEQ ID NO: 1. In one embodiment the mature protein of SEQ ID NO: 1 corresponds the amino acids 22 to 620 of SEQ ID NO: 1.

A glucose oxidase according to the invention is a glucose oxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable glucose oxidases include those of plant, bacterial or fungal origin. Chemically modified or protein engineered mutants are included.

One preferred glucose oxidase for use according to the invention is the glucose oxidase having the sequence of the mature protein of SEQ ID NO:3. In one embodiment the mature protein of SEQ ID NO: 3 corresponds the amino acids 17 to 605 of SEQ ID NO: 3.

The reaction conditions such as concentration of natural rubber, pH, temperature and reaction time may in principle be determined using techniques known in the field for optimizing enzymatic reactions and is completely within the skills of the average practitioner.

The concentration of natural rubber in the reaction mixture is typically in the range of 5-50% DRC, preferably in the range of 10-40% DRC, more preferred in the range of 15-30% DRC and most preferred around 20% such as 20% DRC.

The pH in the reaction mixture should be selected in accordance with the pH preferences of the selected enzyme an is typically in the range of 3.0 to 9.0, preferably in the range of 4.0 to 8.0, more preferred in the range of 5.0 to 7.5, measured at 25° C.

In one embodiment the pH is regulated before the reaction is started whereas in other embodiments the pH is not regulated meaning that the pH in the reaction mixture is determined by the pH of the natural rubber substrate.

The reaction temperature should be selected according to the temperature preferences and temperature stability of the selected enzyme. In general, a higher temperature is preferred to increase the reaction rate however, a higher temperature also provides for a higher inactivation rate for the enzyme, so the skilled person should select the temperature with due consideration of these factors.

The reaction temperature is typically in the range of 10° C. to 75° C., preferably in the range of 20° C. to 60° C., more preferred in the range of 25° C. to 50° C.

The reaction should continue in a sufficient time to achieve the desired degree of epoxidation. The reaction time is typically below 500 minutes such as in the range of 5 minute to 400 minutes, preferably in the range of 30 minutes to 360 minutes, preferably in the range of 60 minutes to 120 minutes.

The degree of epoxidation is in general selected according to the intended use for the epoxidated rubber, and it is understood that the higher degree of epoxidation is selected the longer reaction time and higher enzyme dosage is required to obtain the selected degree of epoxidation.

The degree of epoxidation is typically at least 5%, preferably at least 10%, preferably at least 15%, preferably at least 20%.

The degree of epoxidation may be determined using different methods known in the art, however according to the invention it is preferred to calculate the degree of epoxidation as the percentage of double bonds in the natural rubber starting material that have been epoxidized, measured by NMR.

The epoxidized rubber product prepared using the method according to the invention may in principle be used for the same applications as conventional, chemical epoxidized rubber.

The invention is now further described in examples which are provided for illustrative purposes and should not be considered limiting in any ways.

### PREFERRED EMBODIMENTS

The invention can also be described as the following preferred embodiments:

### Embodiment 1

A method for producing completely or partially epoxidized rubber from natural rubber comprising:
(a) Contacting a natural rubber substrate with one or more enzyme(s) that generates reactive oxygen species in a time period until the desired degree of epoxidation is achieved;
(b) Obtaining the epoxidized rubber.

### Embodiment 2

The method according to embodiment 1, wherein the natural rubber substrate is latex.

### Embodiment 3

The method according to embodiment 1 or 2, wherein the natural rubber substrate is in liquid form.

### Embodiment 4

The method according to embodiment 3, wherein the natural rubber concentration is in the range of 1-50%, preferably 5-30%, preferably 10-25%, preferably around 20% and most preferred 20% Dry Rubber concentrate (DRC)

### Embodiment 5

The method according to any of the preceding embodiments, wherein the one or more enzyme(s) that generate reactive oxygen species are selected among laccases, peroxidases, carbohydrate oxidases and glucose oxidases.

### Embodiment 6

The method according to embodiment 5, wherein the laccase is selected among laccases having an amino acid sequence identity of at least 80% identity, preferably at least 85% identity, preferably at least 90% identity, preferably at least 95% identity, preferably at least 96% identity, preferably at least 97% identity, preferably at least 98% identity, preferably at least 99% identity or 100% identity to the mature protein of SEQ ID NO: 1.

### Embodiment 7

The method according to embodiment 6, wherein the mature protein of SEQ ID NO: 1 corresponds to amino acids 22 to 620 of SEQ ID NO: 1.

### Embodiment 8

The method according to embodiment 5, wherein the peroxidase is selected among peroxidase having an amino acid sequence identity of at least 80% identity, preferably at least 85% identity, preferably at least 90% identity, preferably at least 95% identity, preferably at least 96% identity, preferably at least 97% identity, preferably at least 98% identity, preferably at least 99% identity or 100% identity to the mature protein of SEQ ID NO: 2.

### Embodiment 9

The method according to embodiment 8, wherein the mature protein of SEQ ID NO:2 corresponds to amino acids 19 to 363 of SEQ ID NO: 2.

### Embodiment 10

The method according to embodiment 5, wherein the glucose oxidase is selected among glucose oxidases having an amino acid sequence identity of at least 80% identity, preferably at least 85% identity, preferably at least 90% identity, preferably at least 95% identity, preferably at least 96% identity, preferably at least 97% identity, preferably at least 98% identity, preferably at least 99% identity or 100% identity to the mature protein of SEQ ID NO: 3.

### Embodiment 11

The method of embodiment 10, wherein the mature protein of SEQ ID NO: 3 corresponds the amino acids 17 to 605 of SEQ ID NO: 3.

### Embodiment 12

The method according to any of the embodiments 1 to 5, wherein the enzyme is selected among Denilite IIS, Denilite COLD and Baysolex.

### Embodiment 13

The method according any of the preceeding embodiments, wherein the contacting occurs in the presence or absence of a mediator.

### Embodiment 14

The method according to any of the preceding embodiments, wherein the contacting occurs in the presence of Hydrogen Peroxide (H₂O₂)

### Embodiment 15

The method according to embodiment 14, wherein the Hydrogen peroxide is added in amounts of 1-20%, preferably 5-15%, preferably 7-12%, preferably around 10% and most preferred 10%.

### Embodiment 16

The method according to embodiment 15, wherein the hydrogen peroxide is generated in-situ.

### Embodiment 17

The method according to any of the preceding embodiments, wherein the contacting is done at a pH in the range of 3-8, preferably in the range of 5-7.

### Embodiment 18

The method of embodiment 17, wherein the contacting is done at the pH of the substrate, without any pH adjustment.

### Embodiment 19

The method according to any of the preceding embodiments, wherein the contacting is done at a temperature in the range of 10° C.-70° C., preferably in the range of 20° C.-60° C. most preferred in the range of 25° C.-50° C.

### Embodiment 20

The method according to any of the preceding embodiments, wherein the contacting is carried out for a period between 5 minutes and 400 minutes, preferably in the range of 30 minutes to 360 minutes, preferably in the range of 60 minutes to 120 minutes.

### Embodiment 21

The method according to any of the embodiments 1-20, where the degree of epoxidation is at least 5%, preferably at least 10%, preferably at least 15%, preferably at least 20%, calculated as the percentage of double bonds in the natural rubber starting material that have been epoxidized, measured by NMR.

### Embodiment 22

A completely of partially epoxidized rubber composition prepared using the method according to any of the embodiments 1-21.

### EXAMPLES

### Materials and Methods

### Materials

TABLE 1
Materials used for the natural rubber epoxidation.

| Product | Concentration/Make |
|---|---|
| Natural Rubber 60% Low ammonia | 60% Low ammonia. |
| EZWET TR 3210 | Ceebee chemicals sdn.bhd. |
| (Polyalcohol ethyleneoxide condensates) | 5 pph on DRC |
| Formic acid | 98% |
| Hydrogen peroxide | 50% |
| Methanol | 95% v/v |
| Sodium Carbonate solution | 10% w/v |

### Epoxidation Process

Low or high ammonia centrifuged natural rubber latex diluted to 20% DRC using demineralized or RO water. Stabilizer/wetting agent EZWET TR 3210 was added to the above diluted 20% DRC latex at 5 pph (Parts per hundred) of total DRC (Dry Rubber Content). The diluted latex was stirred slowly for 30 mins at room temperature. Precautions were taken not to create foam while stirring. Diluted latex sample was allocated in glass beaker for epoxidation reaction. Magnetic beads were included in the glass beakers for uniform mixing. 0.75M of 98% formic acid calculated on total DRC was added to the stabilized diluted latex. pH was noted and optionally adjusted. Formic acid was added slowly dropwise to avoid any coagulation of rubber particles. After formic acid addition the temperature was increased to 50° C. Then 50% Hydrogen peroxide at 0.75M on DRC was added to the latex sequentially at regular interval. Enzyme was added to the sample as per the trial plan. In the enzyme treated samples either formic acid or Hydrogen peroxide or both were omitted. The reaction was continued for 4 to 6 hours. For enzyme treated sample Hydrogen peroxide is added after enzyme addition if Hydrogen peroxide is included in the trials. At the end of reaction, the latex was coagulated using 95% methanol. The coagulated epoxidized rubber was washed with 10% sodium carbonate followed by washing with RO water. The coagulated rubber was sheeted to a flat sheet using a roller. Excess water was squeezed out. The rubber sheet was then dried at 50 to 60° C. till rubber sheet appears visually dry.

### Enzymes Used

Denilite IIS: Enzyme preparation comprising laccase (available from Novozymes NS, Bagsvaerd, Denmark)
Denilite COLD: Enzyme preparation comprising peroxidase (available from Novozymes NS, Bagsvaerd, Denmark)
Baysolex VPSP 20019: Enzyme preparation comprising peroxidase (available from Bayer AG, Leverkusen, Germany)

### Methods for Analysis

### 1. FTIR Analysis

TABLE 2
The peak area of interest for epoxidation in FTIR ATR.

| Functional Groups | Peaks |
|---|---|
| C═C | 835-840 |
| Epoxy ring | 870 |
| Epoxy ring | 890 |
| C—O | 1060-1080 |
| Epoxy ring | 1238 |
| C—H bending of CH3 | 1375 |
| C—H stretching | 1447 |

### 2. DSC Analysis

DSC examination of natural rubber (NR) and related cis- and trans-1,4-polyisoprenes is done. The glass transition temperature is a fundamental polymer characteristic, the magnitude of which has a determining influence on the Epoxidation properties of the material. Thus, as the epoxidation increase the temperature also increase. As a thumb rule every 1% mol increase in epoxy group the Tg temperature increases by 1° C.

### 3. NMR Analysis

Nuclear Magnetic resonance analysis of epoxidized rubber was carried out to findout % epoxidation and amount of ring opening post epoxidation.

### Example 1

### Enzymatic Epoxidation of Natural Rubber in Presence of Hydrogen Peroxide and in the Absence of Formic Acid Using Denillite IIS (Laccase)

The epoxidation process was performed in two separate runs with 0.5% and 1% enzyme, without pH regulation before the enzyme addition. The enzyme dosage was on DRC Further testing using FTIR-ATR, DSC or NMR was carried out to quantify degree of epoxidation.

### Results

TABLE 3
NMR results of epoxidized rubber made using Denilite IIS.

| | 1 | 2 |
|---|---|---|
| Denilite IIS dosage | 0.5% | 1% |
| Reaction time | 240 mins | 180 mins |
| % epoxidation | 11.4% | 8.9% |
| % ring opening | 6.3% | 5.9% |

From Table 3 it is observed that formic acid can be successfully replaced from the epoxidation reaction by using Denilite IIS and H₂O₂ combination to the low ammonia centrifuged latex. Denilite IIS and H₂O₂ combination can result 11.4% epoxy group formation.

### Example 2

### Enzymatic Epoxidation of Natural Rubber in Absence of Hydrogen Peroxide and Formic Acid Using Denilite IIS at Various Dosages

Epoxidation of natural rubber was performed using the process described above, except that the pH was adjusted to pH 4.5 before enzyme addition, with various dosages of enzyme as indicated in Table 4.

TABLE 4
Concentration of Denillite IIS.

| | 1 | 2 | 3 | 4 | 5 |
|---|---|---|---|---|---|
| Denilite IIS | 0.5% | 1% | 2.5% | 5% | 10% |

### Results

Peroxidation and ring opening was determined using NMR and the results are disclosed in Table 5.

TABLE 5
NMR results of epoxidized rubber made using Denilite IIS.

| | 1 | 2 | 3 | 4 | 5 |
|---|---|---|---|---|---|
| Reaction time | 120 min | 120 min | 120 min | 60 min | 30 min |
| % epoxidation | 0.9% | 0.8% | 1% | 1.1% | 0.7% |
| % ring opening | 0.4% | 0.3% | 0.5% | 0.6% | 1% |

From Table 5 it is observed that Denilite IIS can form epoxy groups in the natural rubber latex in absence of Hydrogen peroxide and formic acid. Increase in the dosage of Denilite IIS could reduce reaction time of epoxidation for similar percent epoxy group formation.

### Example 3

### Enzymatic Epoxidation of Natural Rubber in Absence of Hydrogen Peroxide and Formic Acid Using Denilite COLD (Peroxidase)

Epoxidation of natural rubber was performed using the process described above, except that the pH was adjusted to pH 4.5 before enzyme addition and hydrogen peroxide was omitted, with various dosages of enzyme as indicated in Table 6.

TABLE 6
Concentration of Denillite COLD.

| | 1 | 2 | 3 |
|---|---|---|---|
| Denilite COLD | 0.5% | 5% | 10% |

### Results

Percent epoxidation and ring opening was determined using NMR analysis.

TABLE 7
NMR results of epoxidized rubber made using Denilite COLD.

| | 1 | 2 | 3 |
|---|---|---|---|
| Reaction time | 60 min | 30 mins | 240 mins |
| % epoxidation | 0.8% | 1.2% | 1.3% |
| % ring opening | 0.4% | 1.0% | 0.8% |

From Table 7 it is observed that Denilite COLD could result epoxy group in natural rubber latex in the absence of hydrogen peroxide and formic acid.

### Example 4

### Enzymatic Epoxidation of Natural Rubber Analysed Through DSC Analysis in the Absence of Formic Acid

Epoxidation of natural rubber was performed using the process described above, where pH was not adjusted before enzyme addition, and hydrogen peroxide was omitted, with various dosages of enzyme as indicated in Table 8.

TABLE 8
Concentration of enzyme.

| | 1 | 2 | 3 |
|---|---|---|---|
| Baysolex VPSP | 1% | | — |
| 20019 (Peroxidase) | | | |
| Denilite IIS | | 1% | — |
| (Laccase) | | | |

### Results

The glass transition temperature for the epoxidated rubber was determined using DCS analysis. Results are shown in Table 9.

TABLE 9
DSC results of epoxidized rubber made using
Denilite IIS & Baysolex VPSP 20019.

| | 1 | | 2 | | 3 | |
|---|---|---|---|---|---|---|
| Reaction time | 240 | mins | 240 | mins | 240 | mins |
| Tg | −63.1° | C. | −62.4° | C. | −63.8° | C. |

In the absence of any acid, Denilite IIS and Baysolex have increased the glass transition temperature (Tg) of the rubber compared with natural (untreated) rubber without epoxy rings.

1. A method for producing completely or partially epoxidized rubber from natural rubber comprising:
(a) Contacting a natural rubber substrate with one or more enzyme(s) that generates reactive oxygen species in a time period until the desired degree of epoxidation is achieved;
(b) Obtaining the epoxidized rubber.

2. The method according to claim 1, wherein the natural rubber substrate is latex.

3. The method according to claim 1, wherein the natural rubber substrate is in liquid form.

4. The method according to claim 3, wherein the natural rubber concentration is in the range of 5-30% Dry Rubber Content (DRC).

5. The method according to claim 1, wherein the one or more enzyme(s) that generate reactive oxygen species are selected among laccases, peroxidases, carbohydrate oxidases and glucose oxidases.

6. The method according to claim 5, wherein the one or more enzyme(s) comprises a laccase having an amino acid sequence identity of at least 80% to the mature protein of SEQ ID NO: 1.

7. The method according to claim 6, wherein the mature protein of SEQ ID NO: 1 corresponds to amino acids 22 to 620 of SEQ ID NO: 1.

8. The method according to claim 5, wherein the one or more enzyme(s) comprises a peroxidase having an amino acid sequence identity of at least 80% the mature protein of SEQ ID NO: 2.

9. The method according to claim 8, wherein the mature protein of SEQ ID NO: 2 corresponds to amino acids 19 to 363 of SEQ ID NO: 2.

10. The method according to claim 5, wherein the one or more enzyme(s) comprises a glucose oxidase having an amino acid sequence identity of at least 80% to the mature protein of SEQ ID NO: 3.

11. The method of claim 10, wherein the mature protein of SEQ ID NO: 3 corresponds the amino acids 17 to 605 of SEQ ID NO: 3.

12. The method according to claim 1, wherein the one or more enzyme(s) comprises Denilite IIS, Denilite COLD or Baysolex.

13. The method according to claim 1, wherein the contacting occurs in the presence of a mediator.

14. The method according to claim 1, wherein the contacting occurs in the presence of Hydrogen Peroxide (H₂O₂)

15. The method according to claim 14, wherein the Hydrogen peroxide is added in amounts of 5-15%.

16. The method according to claim 15, wherein the hydrogen peroxide is generated in-situ.

17. The method according to claim 1, wherein the contacting is done at a pH in the range of 3-8.

18. The method of claim 17, wherein the contacting is done at the pH of the substrate, without any pH adjustment.

19. The method according to claim 1, wherein the contacting is done at a temperature in the range of 10° C.-70° C.

20. The method according to claim 1, wherein the contacting is carried out for a period between 30 minutes to 360 minutes.

21. The method according to claim 1, where the degree of epoxidation is at least 5%, calculated as the percentage of double bonds in the natural rubber starting material that have been epoxidized, measured by NMR.

22. A completely or partially epoxidized rubber composition prepared using the method according to claim 1.
